(19) United States
(12) Patent Application Publication
(10) Pub. No.: US 2013/0316008 A1
(43) Pub. Date: Nov. 28, 2013

(54) MULTICOMPARTMENTALIZED VESICULAR STRUCTURE AND A METHOD FOR FORMING THE SAME

(71) Applicants: Madhavan Nallani, Singapore (SG); Nikodem Tomczak, Singapore (SG); Zhikang Fu, Singapore (SG); Mirjam Ochsner, Singapore (SG)

(72) Inventors: Madhavan Nallani, Singapore (SG); Nikodem Tomczak, Singapore (SG); Zhikang Fu, Singapore (SG); Mirjam Ochsner, Singapore (SG)

(73) Assignee: AGENCY FOR SCIENCE, TECHNOLOGY AND RESEARCH, Singapore (SG)

(21) Appl. No.: 13/814,404

(22) PCT Filed: Aug. 4, 2011
(86) PCT No.: PCT/SG11/00274

§ 371 Date: Jul. 17, 2013

Related U.S. Application Data
(60) Provisional application No. 61/370,847, filed on Aug. 5, 2010.

(51) Int. Cl. A61K 9/14; G01N 33/68

(52) U.S. Cl. CPC A61K 9/146; G01N 33/68
USPC 424/497; 435/375; 436/501; 435/4; 264/4.1; 264/4.6

(57) ABSTRACT

The present invention provides a method for forming a multicompartmentalized vesicular structure comprising an outer block copolymer vesicle and at least one inner block copolymer vesicle, wherein the at least one inner block copolymer vesicle is encapsulated inside the outer block copolymer vesicle. The method comprises forming the at least one inner block copolymer vesicle by any method and adding block copolymers dissolved in a suitable solvent to a dispersion of the at least one inner block copolymer vesicle in an aqueous buffer under conditions that allow the block copolymers to form the outer block copolymer vesicle and encapsulate the at least one inner block copolymer vesicle. A multicompartmentalized vesicular structure and its uses are also provided.

### CROSS-REFERENCE TO RELATED APPLICATION

This application claims the benefit of priority of U.S. Provisional Patent Application No. 61/370,847, filed 5 Aug. 2010, the contents of which being hereby incorporated by reference in its entirety for all purposes.

### TECHNICAL FIELD

The invention relates to a multicompartmentalized vesicular structure, a method for forming the multicompartmentalized vesicular structure and uses of the multicompartmentalized vesicular structure.

### BACKGROUND

Compartmentalization of biochemical processes within membrane-delineated organelles formed by lipids allows for the co-existence of complex reaction pathways in living cells. Besides providing structural support, scaffolding and protection, the differences in selectivity and permeability of the lipid membranes allow for precise control over different biological processes such as the regulation of enzymatic reaction pathways and the synthesis of proteins and nucleic acids. For example, organelles are able to communicate with one another in a specific manner and compartmentalization helps to provide a spatial and temporal separation of many activities inside a cell. Studies have reported encapsulating molecules or substances into compartments to study functions such as the enzymatic activity. However, most of the reported examples relate to single-compartment vesicles made up of detergent bubbles, emulsions, liposomes or polymersomes.

Despite extensive efforts to develop truly compartmentalized vesicular systems, there are relatively few studies tackling the mimicry of intra-cellular events. This is mostly due to the relative thermodynamic and mechanical instability of liposomal compartments. Polymersomes are formed of amphiphilic block co-polymers as building blocks and are an interesting class of materials which are able to self-assemble to form vesicular structures. They offer many advantages compared to liposomes, for example, superior mechanical and colloidal stability under physiological conditions. The size, stability, mechanical, colloidal and physicochemical properties of the polymersomes can be tuned by a proper selection of suitable chemistry and molar masses of the respective “blocks”. These synthetic “molecular containers” have been demonstrated to provide an excellent platform for the encapsulation of molecules (macro or small) and for studying reactions in confined environments. Complex reactions involving enzymes were shown to proceed in an efficient and controlled manner. In a recent attempt to mimic the transport through a cell membrane, a membrane protein (LamB) of E. coli has been reconstituted into polymersomes and the DNA translocation through the receptor was studied.

Although some advances have been made in mimicking the cell and the cellular activities, only single-compartmentalized polymersomes were given attention. Multicompartmentalized “nanoreactors” were not explored primarily due to the challenges in the block copolymer self-assembly to obtain the multicompartmentalized vesicular structures. Additionally, in order to mimic cellular compartments, one should include interfaces with different transport properties to allow control over the transport of molecules in the “nanoreactors”.

Therefore, there is a need to provide for multicompartmentalized vesicular structures to allow more advanced cell mimics and to study cellular organization.

### SUMMARY

In a first aspect, there is provided a method for forming a multicompartmentalized vesicular structure comprising an outer block copolymer vesicle and at least one inner block copolymer vesicle, wherein the at least one inner block copolymer vesicle is encapsulated inside the outer block copolymer vesicle. The method comprises:
(a) forming the at least one inner block copolymer vesicle by any method; and
(b) adding block copolymers dissolved in a suitable solvent to a dispersion of the at least one inner block copolymer vesicle in an aqueous buffer under conditions that allow the block copolymers to form the outer block copolymer vesicle and encapsulate the at least one inner block copolymer vesicle.

In a further aspect, there is provided a multicompartmentalized vesicular structure obtained according to the present method.

In another aspect, there is provided a multicompartmentalized vesicular structure comprising an outer block copolymer vesicle and at least one inner block copolymer vesicle, wherein the at least one inner block copolymer vesicle is encapsulated inside the outer block copolymer vesicle, and wherein the outer block copolymer vesicle, the at least one inner block copolymer vesicle, or both include at least one substance encapsulated inside the respective vesicle.

In a further aspect, use of the present multicompartmentalized vesicular structure for selective encapsulation of different substances, wherein the inner block copolymer vesicle encapsulates a first substance and the outer block copolymer vesicle encapsulates a second substance and the inner block copolymer vesicle is provided.

In another further aspect, it is provided a method of releasing or delivering substances encapsulated in the respective inner block copolymer vesicle and outer block copolymer vesicle of the present multicompartmentalized vesicular structure, wherein the method comprises contacting the multicompartmentalized vesicular structure with a cell so that the multicompartmentalized vesicular structure is taken up into the cell.

In one aspect, a pharmaceutical composition is provided, comprising the present multicompartmentalized vesicular structure and a pharmaceutically acceptable carrier.

In another apsect, there is provided an in-vitro method of identifying a compound capable of forming a complex with a membrane receptor protein. The method comprises:
(a) providing the present multicompartmentalized vesicular structure, wherein the membrane associated protein is the membrane receptor protein;
(b) contacting the multicompartmentalized vesicular structure with a candidate compound suspected to be capable of forming a complex with the membrane receptor protein; and
(c) detecting the said complex formation.

In yet another aspect, it is provided an in-vitro method of identifying a compound capable of modulating the function of an ion channel protein, wherein the ion channel protein is capable of allowing a known ion to pass. The method comprises:
(a) providing the present multicompartmentalized vesicular structure, wherein the membrane protein is the ion channel protein;
(b) contacting the multicompartmentalized vesicular structure with a candidate compound suspected to modulate the function of the ion channel protein; and
(c) detecting the passage of ions into or out of the multicompartmentalized vesicular structure.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows a scheme of the present method to form the present multicompartmentalized vesicular structure having vesicles encapsulating different proteins.

FIG. 2 shows TEM images of individual and multicompartmentalized polymersomes: (a) individual ABA polymersomes; (b) individual PS-PIAT polymersomes; (c) multicompartmentalized polymersomes encapsulating GFP and Cy5-IgG. Three populations of vesicles can be clearly seen; two populations of individual ABA (thin arrow) and PS-PIAT polymersomes (thick arrow), and a population of multicompartmentalized polymersomes (inset).

FIG. 3 shows DLS data showing size distribution of (a) individual ABA polymersomes (average 100 nm); (b) individual PS-PIAT polymersomes (average 145 nm); (c) multicompartmentalized polymersomes (average 160 nm).

FIG. 4 shows scanning electron microscopy (SEM) and fluorescence images of vesicles: (a) SEM image of ABA vesicles; (b) fluorescence image of ABA vesicles with GFP (excitation wavelength: 470 nm); (c) SEM image of PS-PIAT vesicles; (d) fluorescence image of PS-PIAT vesicles with Cy5-IgG (excitation wavelength: 640 nm).

FIG. 5 shows (a) SEM and (b) fluorescence image of the present multicompartmentalized vesicles (ABA containing calcein and PS-PIAT containing Cy5-labeled IgG). Green spots correspond to GFP encapsulated in individual ABA polymersomes while red spots correspond to Cy5-IgG encapsulated in individual PS-PIAT polymersomes. Spots with colocalized green and red emission show up in yellow and correspond to multicompartmentalized polymersomes.

FIG. 6 shows scanning confocal fluorescence image of multicompartmentalized polymersomes. Green spots correspond to non-encapsulated calcein, while red spots correspond to Cy5-IgG encapsulated in PS-PIAT polymersomes. Spots with co-localized green and red emission show up in yellow and correspond to multicompartmentalized polymersomes.

FIG. 7 shows flow cytometry data of the present multicompartmentalized polymersomes: (a) Dot plot from dispersions of multicompartmentalized polymersomes without any encapsulated fluorophores. FSC/SSC gating was applied (region R1) to exclude larger particles and noise. (b) Dot plot of FL1 (530±30 nm for calcein detection) against FL4 (650 nm for Cy5-IgG detection) after the gating. (c) Dot plot of multicompartmentalized polymersomes encapsulating calcein (ABA compartment) and Cy5-IgG (PS-PIAT compartment) where gate R1 was applied to the FSC/SSC dot plot. Calcein was used instead of GFP as it gives a larger signal. (d) Dot plot of FL1/FL4 after gating, clearly showing in the upper right quadrant the presence of polymersomes encapsulating both calcein and Cy5-IgG.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

In one aspect, there is provided a method for forming a multicompartmentalized vesicular structure. The multicompartmentalized vesicular structure comprises an outer block copolymer vesicle and at least one inner block copolymer vesicle. The at least one inner block copolymer vesicle is encapsulated inside the outer block copolymer vesicle.

In the present context, polymersomes are vesicles with a polymeric membrane, which are typically, but not necessarily, formed from the self-assembly of dilute solutions of amphiphilic block copolymers, which can be of different types such as diblock and triblock. Polymersomes may also be formed of tetrablock or pentablock copolymers. For triblock copolymers, the central block is often shielded from the environment by its flanking blocks, while diblock copolymers self-assemble into bilayers, placing two hydrophobic blocks tail-to-tail, much to the same effect. In most cases, the vesicular membrane has an insoluble middle layer and soluble outer layers. The driving force for polymersome formation by self-assembly is considered to be the microphase separation of the insoluble blocks, which tend to associate in order to shield themselves from contact with water. Polymersomes possess remarkable properties due to the large molecular weight of the constitutent copolymers. Vesicle formation is favored upon an increase in total molecular weight of the block copolymers. As a consequence, diffusion of the (polymeric) amphiphiles in these vesicles is very low compared to vesicles formed by lipids and surfactants. Owing to this less mobility of polymer chains aggregated in vesicle structure, it is possible to obtain stable polymersome morphologies. In the context of the present invention, a vesicle does not refer to a structure comprising fats, oils, waxes, cholesterol, sterols, fat-soluble vitamins (such as vitamins A, D, E and K), monoglycerides, diglycerides, and phospholipids. Unless expressly stated otherwise, the term “polymersome” and “vesicle”, as used hereinafter, are taken to be analogous and may be used interchangably.

In various embodiments, the block copolymer forming the outer block copolymer vesicle and the block copolymer forming the at least one inner block copolymer vesicle are the same or different. The outer block copolymer vesicle may be a polymersome formed of an amphiphilic diblock, triblock, tetrablock or pentablock copolymer. In certain embodiments, the outer block copolymer vesicle is a polymersome formed of a diblock copolymer. The inner block copolymer vesicle may be a polymersome formed of an amphiphilic diblock, triblock, tetrablock or pentablock copolymer. In certain embodiments, the inner block copolymer vesicle is a polymersome formed of a triblock copolymer. In various embodiments, the at least one inner block copolymer vesicle includes at least two block copolymer vesicles that are the same or different.

In some embodiments, each of the block copolymer of the outer vesicle and the inner vesicle includes a polyether block such as a poly(oxyethylene) block, a poly(oxypropylene) block, and a poly(oxybutylene) block. Further examples of blocks that may be included in the copolymer include, but are not limited to, poly(acrylic acid), poly(methyl acrylate), polystyrene, poly(butadiene), poly(2-methyloxazoline), poly(dimethyl siloxane), poly(L-isocyanoalanine(2-thiophen-3-yl-ethyl)amide), poly(e-caprolactone), poly(propylene sulphide), poly(N-isopropylacrylamide), poly(2-vinylpyridine), poly(2-(diethylamino)ethyl methacrylate), poly(2-(diisopropylamino)ethylmethacrylate), poly(2-(methacryloyloxy)ethylphosphorylcholine) and poly(lactic acid). Examples of suitable outer vesicles and inner vesicles include, but are not limited to, poly(ethyl ethylene)-b-poly(ethylene oxide) (PEE-b-PEO), poly(butadiene)-b-poly(ethylene oxide) (PBD-b-PEO), poly(styrene)-b-poly(acrylic acid) (PS-b-PAA), poly(ethylene oxide)-poly(caprolactone) (PEO-b-PCL), poly(ethylene oxide)-poly(lactic acid) (PEO-b-PLA), poly(isoprene)-poly(ethylene oxide) (PI-b-PEO), poly(2-vinylpyridine)-poly(ethylene oxide) (P2VP-b-PEO), poly(ethylene oxide)-poly(N-isopropylacrylamide) (PEO-b-PNIPAm), poly(ethylene glycol)-poly(propylene sulfide) (PEG-b-PPS), poly(methylphenylsilane)-poly(ethylene oxide) (PMPS-b-PEO-b-PMPS-b-PEO-b-PMPS), poly(2-methyloxazoline)-b-poly(dimethylsiloxane)-b-poly(2-methyloxazoline) (PMOXA-b-PDMS-b-PMOXA), poly(2-methyloxazoline)-b-poly(dimethylsiloxane)-b-poly(ethylene oxide) (PMOXA-b-PDMS-b-PEO), poly[styrene-b-poly(L-isocyanoalanine(2-thiophen-3-yl-ethyl)amide)](PS-b-PIAT), poly(ethylene oxide)-b-poly(propylene sulfide)-b-poly(ethylene oxide) (PEO-b-PPS-b-PEO) and a poly(ethylene oxide)-poly(buylene oxide) (PEO-b-PBO) block copolymer. A block copolymer can be further specified by the average number of the respective blocks included in a copolymer. Thus PS_{M}-PIAT_{N} indicates the presence of polystyrene blocks (PS) with M repeating units and poly(L-isocyanoalanine(2-thiophen-3-yl-ethyl)amide) (PIAT) blocks with N repeating units. M and N are independently selected integers, which may for example be selected in the range from about 5 to about 95. Thus PS₄₀-PIAT₅₀ indicates the presence of PS blocks with an average of 40 repeating units and of PIAT blocks with an average of 50 repeating units.

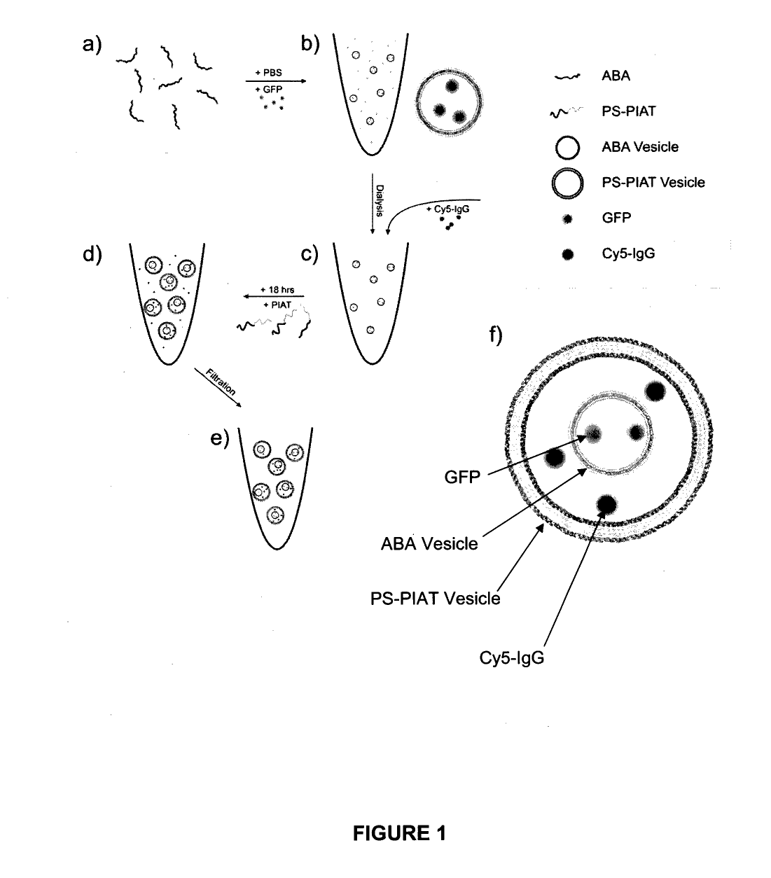

By “encapsulated” it is meant that, as illustrated in FIG. 1f, the inner vesicle (e.g., ABA) is completely contained inside the outer vesicle (e.g., PS-PIAT) and is surrounded by the vesicular membrane of the outer vesicle. The confined space surrounded by the vesicular membrane of the outer vesicle forms one compartment. In the illustration, the outer vesicle encapsulates Cy5-IgG and the inner vesicle, in turn encapsulates GFP. The confined space surrounded by the vesicular membrane of the inner vesicle forms another compartment. Hence, the multicompartmentalized vesicular structure defined by the vesicular membrane of the outer vesicle illustrated in FIG. 1f contains two separate compartments. Such dual-compartment structure may also be referred to as double encapsulation.

In various embodiments, the method includes forming the at least one inner block copolymer vesicle and adding block copolymers dissolved in a suitable solvent to a dispersion of the at least one inner block copolymer vesicle in an aqueous buffer under conditions that allow the block copolymers to form the outer block copolymer vesicle and encapsulate the at least one inner block copolymer vesicle.

The solvent for dissolving the block copolymer of the outer vesicle may be include, but is not limited to, tetrahydrofuran (THF), chloroform, ethanol, dimethylsulfoxide (DMSO), dimethylformamide (DMF), toluene, dioxane, or water. In some embodiments, the solvent may be an ionic liquid. Examples of the ionic liquid include, but are not limited to, 1-ethyl-3-methylimidazolium tetrafluoroborate, N-butyl-4-methylpyridinium tetrafluoroborate, 1,3-dialkylimidazolium-tetrafluoroborate, 1,3-dialkylimidazolium-hexafluoroborate, 1-ethyl-3-methylimidazolium bis(pentafluoroethyl)phosphinate, 1-butyl-3-methyl-imidazolium tetrakis(3,5-bis(trifluoromethylphenyl)borate, tetrabutyl-ammonium bis(trifluoromethyl)imide, ethyl-3-methylimidazolium trifluoromethanesulfonate, 1-butyl-3-methylimidazolium methylsulfate, 1-n-butyl-3-methylimidazolium ([bmim]) octylsulfate, and 1-n-butyl-3-methylimidazolium tetrafluoroborate.

The aqueous buffer may be a buffered solution. In various embodiments, the buffered solution is a buffered saline solution such as phosphate buffered saline (PBS), Tris buffered saline (TBS), Hank's balanced salt solution (HBSS), Earle's balanced salt solution (EBSS), Standard saline citrate (SSC), HEPES-buffered saline (HBS), and Grey's balanced salt solution (GBSS). Alternatively, or additionally, the buffer may be buffers with detergents, such as but not limited to, buffers formed of sodium carbonate, N-(2-Hydroxyethyl)-piperazine-N′-ethanesulfonic acid (HEPES), 3-(N-Morpholino)-propanesulfonic acid, Tris(hydroxymethyl)-aminomethane (Tris) and phosphates.

The block copolymers may be allowed to form the outer block copolymer vesicle by incubating the reaction mixture for at least 10 min, such as about 1 hour, about 6 hours, about 12 hours, about 24 hours, or more than 30 hours.

The at least one inner block copolymer vesicle may be formed by any method. In certain embodiments, the at least one inner block copolymer vesicle is formed by dissolving block copolymers in a suitable solvent, drying the solution to obtain a polymer film, and rehydrating the polymer film of in an aqueous buffer.

In various embodiments, the outer block copolymer vesicle, the at least one inner block copolymer vesicle, or both include at least one substance encapsulated inside the respective vesicle. A multicompartmentalized vesicular structure including an outer block copolymer vesicle and at least one inner block copolymer vesicle, whereby the at least one inner block copolymer vesicle is encapsulated inside the outer block copolymer vesicle, and whereby the outer block copolymer vesicle, the at least one inner block copolymer vesicle, or both include at least one substance encapsulated inside the respective vesicle is contemplated.

The block copolymer of the inner vesicle is able to self-assemble into a vesicular structure with a bilayer shell when added to a buffer at a suitable concentration. The block copolymer should not interact with the encapsulated substance in any way that will prevent it from forming a vesicular structure. The bilayer of the vesicular structure formed by the inner block copolymer is able to prevent significant diffusion of encapsulated substance across the vesicular membrane. The size of the inner vesicle is smaller than the size of the outer vesicle and may be tuned by extrusion, for example.

The block copolymer of the outer vesicle is able to self-assemble into a vesicular structure with a bilayer shell when added to a buffer at a suitable concentration. The block copolymer should not interact with the encapsulated substance and the inner block copolymer vesicle including its encapsulated substance in any way that will prevent the block copolymer of the outer vesicle from forming a vesicular structure. The bilayer of the vesicular structure formed by the outer block copolymer is able to prevent significant leakage of encapsulated substance across the vesicular membrane. Further, the solvent in which the block copolymers of the outer vesicle is dissolved should not interact with inner vesicle in such a way that will cause the outer vesicle to lose its vesicular structure or lead to the release of the encapsulated substance in the inner vesicle. The outer vesicle should be sufficiently large to encapsulate the extruded inner vesicle.

The at least one inner block copolymer vesicle encapsulating the at least one substance may be obtained by dissolving block copolymers in a suitable solvent, drying the solution to obtain a polymer film, and rehydrating the polymer film in an aqueous buffer containing the at least one substance.

The outer block copolymer vesicle encapsulating the at least one substance may be obtained by adding block copolymers dissolved in a suitable solvent to a dispersion of the at least one inner block copolymer vesicle in an aqueous buffer under conditions that allow the block copolymers to form the outer block copolymer vesicle and encapsulate the at least one inner block copolymer vesicle. The at least one substance is added (i) to the solution of the block copolymers or (ii) the dispersion of the at least one inner block copolymer vesicle before adding the solution of the block copolymers to encapsulate the substance.

In various embodiments, the at least one substance is selected from the group consisting of organic molecules, biomolecules and ions. In certain embodiments, the biomolecules include proteins, lipids, carbohydrates, and nucleic acids. In alternative embodiments, the at least one substance is a marker substance selected from the group consisting of fluorophores, chromophores, radiomarkers, fluorescent proteins, enzymes, and fluorophore-, chromophore- or radio-labelled proteins.

In various embodiments, the outer block copolymer vesicle, the at least one inner block copolymer vesicle, or both include at least one molecule that modulates vesicular membrane permeability. The molecule may be present in the vesicular membrane. In certain embodiments, the molecule is selected from the group of transmembrane proteins, membrane associated proteins, and lipids. In some embodiments, the molecule is a transporter molecule or ion channel.

The present multicompartmentalized vesicular structure may be used for selective encapsulation of different substances in the respective vesicle. The block copolymer forming the outer block copolymer vesicle and the block copolymer forming the at least one inner block copolymer vesicle are chosen to be of different composition to form the vesicular multicompartments and membranes. By choosing the different composition, one can control the location and action of the encapsulated substances in the multicompartmentalized structure, for example, control over the transport of the encapsulated substances between the individual compartments within the multicompartmentalized structure. The inner block copolymer vesicle encapsulates a first substance and the outer block copolymer vesicle encapsulates a second substance and the inner block copolymer vesicle. The present multicompartmentalized vesicular structure may thus be used for controlled release or delivery of the different substances encapsulated in the respective inner block copolymer vesicle and outer block copolymer vesicle. The multicompartmentalized vesicular structure may be contacted with a cell so that the multicompartmentalized vesicular structure is taken up into the cell. The cell may be in vitro. Alternatively, the cell is in vivo and the multicompartmentalized vesicular structure is administered to a subject, for example a human. The controlled release or delivery may be triggered by a stimulant such as light, temperature, pH, electric field, magnetic field, bacteria, viruses, pathogens, or chemical compounds.

A pharmaceutical composition including the present multicompartmentalized vesicular structure and a pharmaceutically acceptable carrier is also contemplated. In some embodiments, the encapsulated substance includes a pharmaceutical or chemical compound such as, but are not limited to, (8S,10S)-10-(4-amino-5-hydroxy-6-methyl-tetrahydro-2H-pyran-2-yloxy)-6,8,11-tri hydroxy-8-(2-hydroxyacetyl)-1-methoxy-7,8,9,10-tetrahydrotetracene-5,12-dione (Doxorubicine; anticancer drug), {(1Z)-5-fluoro-2-methyl-1-[4-(methylsulfinyl)benzylidene]-1H-indene-3-yl}acetic acid (sulindac); of (+)-(S)-2-(6-methoxynaphthalen-2-yl)propanoic acid (naproxen; NSAID), 2-(3-benzoyl phenyl)propionic acid (ketoprofen; NSAID), 4-chloro-N-(2-furylmethyl)-5-sulfamoylanthranilic acid (furosemide; used for the treatment of congestive heart failure and edema), and N-(2,3-dimethylphenyl)anthranilic acid (mefenamic acid; NSAID).

Multicompartmentalized vesicles according to the invention can be used as a carrier for a drug, a marker or other matter to be administered to a human or animal body. The vesicle, as well as substances encapsulated therein, can be administered to a cell, an animal or a human patient per se, or in a pharmaceutical composition. Exemplary routes include, but are not limited to, oral, transdermal, and parenteral delivery. The pharmaceutical composition of the present invention including the multicompartmentalized vesicles offers great advantages for the administration of pharmaceutical compounds. The vesicular membrane may be tuned to be pH sensitive and the encapsulated drug will not be released, for example, by the gastric acid (pH below about 5) in the stomach. The drug is protected so that no degradation or modification of the active compound will take place. Once the drug passes to the intestine, the pH value of the environment raises to above about 5 and the drug can be released.

In another aspect, an in vitro method of identifying a compound capable of forming a complex with a membrane receptor protein is provided. The method includes providing the present multicompartmentalized vesicular structure whereby the membrane associated protein is the membrane receptor protein, contacting the multicompartmentalized vesicular structure with a candidate compound suspected to be capable of forming a complex with the membrane receptor protein, and detecting the said complex formation. The multicompartmentalized vesicular structure may be immobilized on a surface.

In some embodiments a membrane protein is associated with/integrated into the vesicular membrane that is intended to be subject to an assay or a screening method. As an example, it may be desired to identify a compound that is capable of modulating, such as stimulating or inhibiting, including blocking, a membrane protein. The respective membrane protein, which may be any membrane protein, may be expressed and associated with/integrated into the membrane of the present vesicular structure. Where a measurable effect of the membrane protein, e.g. a cellular response, is known the required components to achieve such a response may be integrated into the vesicle. In embodiments where the membrane protein is responsive to external molecules, it may be termed a receptor protein. A respective molecule from the ambience may form a complex with the receptor protein. Thereby the receptor protein may undergo a change, such as a conformational change, from an active state to an inactive state and vice versa. As a first step it may be desired to identify a compound that is able to form a complex with such a receptor protein. Once such a complex is identified a cellular effect may be analysed, for example by expressing an effector protein and integrating the same into the vesicle. A stimulation or inhibition of the effector protein may then be determined.

The present multicompartmentalized vesicle with an associated or integrated membrane protein may in some embodiments be used for the in vitro screening for potential compounds that are useful for modulating the function of the membrane protein, including the simultaneous screening of compound libraries on multiple-well microplates using automated work stations.

In some embodiments it is determined whether the formation of a complex between a candidate compound and a membrane receptor protein occurs. In such an embodiment an immobilized vesicle may be provided which has an associated/integrated membrane receptor protein. The candidate compound is brought in contact with the vesicle and thereby with the membrane receptor protein. The vesicle may for example be provided in aqueous solution to which the candidate compound is added. Further the complex formation is detected. In some embodiments the candidate compound has a label, such as a radioactive label or a photoactive, e.g. a luminescent, label, allowing the detection of a complex after for example a washing step, which is a standard procedure in the art. Further examples of determining the formation of a complex as defined above, may for instance rely on spectroscopic, photochemical, photometric, fluorometric, radiological, enzymatic or thermodynamic means. An example for a spectroscopic detection method is fluorescence correlation spectroscopy. A photochemical method is for instance photochemical cross-linking. The use of photoactive, fluorescent, radioactive or enzymatic labels respectively are examples for photometric, fluorometric, radiological and enzymatic detection methods. An example for a thermodynamic detection method is isothermal titration calorimetry. Some of these methods may include additional separation techniques such as electrophoresis or HPLC. Examples for the use of a label comprise a compound as a probe or an antibody with an attached enzyme, the reaction catalysed by which leads to a detectable signal. An example of a method using a radioactive label and a separation by electrophoresis is an electrophoretic mobility shift assay.

In some embodiments a respective method may be an in vitro method of identifying a compound that is capable of modulating the function of a (cellular) receptor protein. The receptor protein is capable of inducing a known cellular response. The method includes providing a multicompartmentalized vesicle including a membrane protein. This membrane protein is associated to the membrane of vesicle. The membrane protein is a cellular receptor protein, which is the cellular receptor protein that is capable of inducing the known cellular response. The method includes contacting the vesicle with a candidate compound. The candidate compound is suspected to modulate the function of the cellular receptor protein. The method also includes detecting the known cellular response.

A respective membrane protein integrated into the membrane of the multicompartmentalized vesicle may also be an ion channel, an ion transporter or a ionotropic receptor. It may be determined whether a candidate compound is capable of modulating the function of an ion channel or ion transporter protein. In such an embodiment the vesicle may have in its interior phase an indicator that is sensitive to the presence of the ion, which the ion channel/transporter protein is capable of allowing to pass. In some embodiments the ion channel or transporter is selective for this ion. The vesicle with the ion channel/transporter protein may be contacted with a candidate compound. The passage of ions into or out of the vesicle may then be detected, for instance by means of an indicator.

In some embodiments one of the above methods of identifying a (candidate) compound may also include comparing the results of detecting, including measuring, the cellular response. The result may for example be compared to a control measurement. For a respective control measurement a compound may be used that is known not to affect the function of the cellular receptor protein. In typical embodiments an altered cellular response as compared to the control measurement indicates that the candidate compound is capable of modulating the function of the cellular receptor protein.

In a further aspect, an in vitro method of identifying a compound capable of modulating the function of an ion channel protein, whereby the ion channel protein is capable of allowing a known ion to pass is provided. The method comprises providing the present multicompartmentalized vesicular structure whereby the membrane protein is the ion channel protein, contacting the multicompartmentalized vesicular structure with a candidate compound suspected to modulate the function of the ion channel protein, and detecting the passage of ions into or out of the multicompartmentalized vesicular structure. The multicompartmentalized vesicular structure may be immobilized on a surface.

In embodiments where the function of an ion channel, ion pump, ion transporter or ionotropic receptor is to be analysed, a control experiment may be used to analyse the integrity of the present vesicle used. Leakage of ions across the polymer membrane of the vesicle may easily be detected by means of an indicator sensitive to ions, including sensitive to the ion for which the respective ion channel, ion pump or ion transporter is specific.

In some embodiments a respective method may be an in vitro method of identifying a portion, e.g. a domain or an amino acid, of a membrane protein, e.g. a receptor, ion channel or ion transporter protein, that is of particular relevance to the function of the membrane protein. As an illustrative example, mutants of a membrane protein of interest may be compared using the present vesicles under comparable or the same conditions. In some embodiments a plurality of such mutants may be analysed in parallel. The membrane proteins may for example be compared in terms of the capability of carrying out their biological function, e.g. amount of ions allowed to pass, including their sensitivity to conditions of the ambience (e.g. temperature, pH, ion concentration etc.) in carrying out their biological function. In one embodiment a library of membrane proteins, including a library of variants of a single protein, produced by in vitro synthesis, may be examined.

For some embodiments of a method of identifying a candidate compound according to the invention, compounds may be used in form of a library. Examples of such libraries are collections of various small organic molecules, chemically synthesized as model compounds, or nucleic acid molecules containing a large number of sequence variants. A method of identifying a compound according to the invention may be carried out as a screening method, including a high-throughput method. In a respective method a library of compounds may for example be screened to identify candidate compounds capable of complex formation with a membrane protein such as a receptor protein. In embodiments where a plurality of candidate compounds are analysed according to a method of the present invention in order to identify a compound capable of modulating a function of a membrane protein, such an embodiment may typically called a screening process. These candidate compounds may be analysed independent from each other, e.g. concurrently, consecutively or in any way out of phase. In some embodiments any number of steps of analysing a plurality of candidate compounds may for example be carried out automatically—also repeatedly, using for instance commercially available robots. For such purposes any number of automation devices may be employed, for instance an automated read-out system, a pipetting robot, a rinsing robot, or a fully automated screening system. As an illustrative example, the process may be an in vitro screening process, for example carried out in multiple-well microplates (e.g. conventional 48-, 96-, 384- or 1536 well plates) using one or more automated work stations. Hence, in some embodiments the invention provides a process of high-throughput screening.

A multicompartmentalized vesicle according to the invention can in some embodiments be immobilized to a surface and thus serve as a stable alternative for a liposome and can be incorporated into a surface-bound architecture that includes the vesicle anchored onto a supporting matrix via an amphiphlic polymer. This architecture could be used as a biochemical reaction chamber to carry out detailed functional analysis of membrane proteins. In particular, the in vitro insertion of membrane proteins into vesicular/spherical architectures enhances the amount of “active material”, such as protein moieties. This allows optimization of the signal-to-noise ratio in sensing applications and is of potential interest for structure-resolution approaches, based on Infrared technologies. Other applications of this architecture include but are not limited to, a bio-chemical sensor based on binding to a specific receptor. As an illustrative example, an odor based sensor may include a plurality of immobilized vesicles that carry a respective odor receptor. A vesicle of the invention may also be immobilized onto a substrate that is to be used in vivo. The vesicle may include in its interior a pharmaceutically active compound that is released upon a certain tissue signal, thereby allowing triggered drug delivery as discussed above. An architecture based on one or more immobilized vesicles may also be used for food or water quality testing. As a further example, one or more immobilized vesicles may be used in pathogen detection. As a further example, a dye or nanocrystals such as quantum dots may be incorporated within the interior of an immobilized vesicle, thereby for example facilitating detection.

In one embodiment, a multicompartmentalized vesicular structure based on sequential self-assembly of two different block copolymers is shown in FIG. 1. Poly[-(2-methyloxazoline)-poly-(dimethylsiloxane)-poly-(2-methyloxazoline)](PMOXA₁₂-PDMS₅₅-PMOXA₁₂) (“ABA”) and poly[styrene-b-poly-(L-isocyanoalanine(2-thiophen-3-yl-ethyl)amide)](PS₄₀-PIAT₅₀) (“PS-PIAT”) are employed. The sequential self-assembly of these copolymers and purification of the resulting polymersomes result in a vesicle-in-vesicle structure including (at least) two compartments delineated by structurally different polymer membranes. ABA polymersomes, which possess a tightly packed membrane structure that limits transport across the membrane were designated as the inner vesicles, while PS-PIAT polymersomes, which have a semi-permeable membrane that allows the diffusion of small molecules were designated as the outer vesicles.

ABA polymersomes were first formed through film rehydration by dissolving ABA copolymers in ethanol, drying the solution under a stream of nitrogen to obtain a polymer film, and rehydrating the polymer film in phosphate buffered saline (PBS) containing biotin conjugated green fluorescent protein (GFP). Subsequently, PS-PIAT polymersomes were formed in the presence of ABA polymersomes using the direct dissolution method by adding PS-PIAT copolymers dissolved in tetrahydrofuran (THF) to a dispersion of ABA polymersomes and cyanine-5 conjugated Immunoglobin G (Cy5-IgG). A multicompartmentalized vesicular structure of FIG. 1f is thus obtained.

Various embodiments allow formation of compartmentalized vesicular structures made solely of self-assembled block copolymers. This is a significant improvement over currently available technology, as copolymer vesicles offer superior colloidal and mechanical stability, allowing for greater ease of use and long term applications and studies. Additionally, there is a large spectrum of possible polymer structures that can be used in the self-assembly process, allowing precise control over membrane properties that can be targeted towards specific applications. Polymersomal technology is also compatible with techniques related to the study of transmembrane proteins. In summary, a versatile method for the fabrication of complex compartmentalized vesicular structures is provided.

In order that the invention may be readily understood and put into practical effect, particular embodiments will now be described by way of the following non-limiting examples.

### EXAMPLES

A simple method based on sequential self-assembly to prepare multicompartmentalized vesicular structure is presented. Two different block copolymers were employed for the self assembly, namely PMOXA₁₂-PDMS₅₅-PMOXA₁₂ (poly[-(2-methyloxazoline)-poly-(dimethylsiloxane)-poly-(2-methyloxazoline)]) (ABA), and PS₄₀-PIAT₅₀ (poly[styrene-b-poly(L-isocyanoalanine(2-thiophen-3-yl-ethyl)amide)]) (PS-PIAT). The sequential self-assembly of these copolymers and purification of the resulting polymersomes result in a vesicle-in-vesicle structure comprising (at least) two compartments delineated by structurally different polymer membranes. ABA polymersomes, which possess a tightly packed membrane structure that limits transport across the membrane were designated as the inner vesicles, while PS-PIAT polymersomes, which have a semi-permeable membrane that allows the diffusion of small molecules were designated as the outer vesicles. Using a combination of procedures for the self-assembly of ABA and PS-PIAT polymersomes, a method of encapsulating ABA polymersomes within PS-PIAT polymersomes to form a multicompartmentalized vesicular structure is illustrated.

### Materials and Methods

Poly[(2-methyloxazoline)-poly-(dimethylsiloxane)-poly-(2-methyloxazoline)] (PMOXA₁₂-PDMS₅₅-PMOXA₁₂) ABA triblock copolymer (M_{w}˜11 kDa) was obtained from the group of Prof. Wolfgang Meier, University of Basel, Switzerland. Poly[styrene-b-poly(L-isocyanoalanine (2-thiophen-3-yl-ethyl)amide)](PS₄₀-PIAT₅₀) diblock copolymer (M_{w}˜6 kDa) was obtained from the group of Prof. Jan van Hest, Netherlands. Biotin conjugated green fluorescent protein (GFP-Biotin) (M_{w}˜27 kDa) was obtained from Dr. Emma van Loung, Institute of Materials Research and Engineering, Singapore. Cyanine-5 conjugated Immunoglobin G (Cy5-IgG) (M_{w}˜150 kDa) was bought from Chemicon International. Calcein (M_{w}-622.55 Da) and phosphate buffered saline (PBS, 10×, pH 7.4) were purchased from Sigma Aldrich (Singapore) and Invitrogen (Gibco), respectively. Absolute Ethanol was bought from Fisher (UK) and tetrahydrofuran (THF) was purchased from Tedia (USA, Ohio).

### Preparation of (PMOXA₁₂-PDMS₅₅-PMOXA₁₂) Polymersome (Inner Vesicle)

(PMOXA₁₂-PDMS₅₅-PMOXA₁₂) ABA triblock copolymer polymersomes were prepared using the film rehydration method. 5.0 mg of ABA copolymers were dissolved in 200 μl of ethanol and evaporated slowly under a stream of nitrogen in a conical bottom schlenk tube to form a polymer film. The film was dried for at least 4 h under a constant nitrogen stream. 1.0 ml of 10% GFP (or 30 mM calcein) in PBS was added to the tube and stirred gently for at least 18 h to rehydrate the film and allow spontaneous formation of the polymersomes, obtaining a uniformly turbid dispersion (FIG. 1b). The resulting dispersion was extruded several times through 0.45 μm and 0.22 μm syringe filters (PVDF, Millex) successively to obtain the monodisperse vesicles. They were further extruded through a 100 nm filter using a mini-extruder (Avanti Polar Lipids). Non-encapsulated GFP (or calcein) molecules were removed by dialysis (MWCO 50 kDa, Spectra/Por® 7, Spectrum Laboratories) against PBS for at least 24 hours.

### Preparation of (PS₄₀-PIAT₅₀) Polymersomes (Outer Vesicle)

(PS₄₀-PIAT₅₀) AB diblock copolymer polymersomes were prepared by direct dissolution. 0.5 mg of PS₄₀-PIAT₅₀ was dissolved in 500 μl of THF and added into 2.5 ml of PBS containing 60 μg of Cy5-IgG. The mixture was left at room temperature for at least 12 h. In order to remove the non-encapsulated Cy5-IgG molecules, the dispersion was passed 8 times through centrifugal filters with 0.1 μm cut-off (Ultrafree-MC (PVDF), Amicon Millipore) at 3,000 rpm for 10 min each time (MiniSpin® plus, Eppendorf).

### Preparation of Multicompartmentalized Vesicular Structure

For the formation of multicompartmentalized vesicular structures, 500 μl of purified ABA polymersomes solution was added to 2.0 ml of PBS containing 60 μg of Cy5-IgG. (FIG. 1c). 500 μl of 1 mg/ml PS₄₀-PIAT₅₀ in THF was added dropwise to the above solution (FIG. 1d). After 12 h of incubation, the dispersion was purified by using centrifugal filters with 0.1 μm cut-off, as mentioned above.

### TEM Measurements

Transmission electron microscopy (TEM) imaging was performed with a Philips CM300 FEGTEM. The samples were prepared by dispensing a 15 μl drop of vesicle dispersion on a copper grid followed by the removal of excess solution with filter paper after 30 min of incubation.

### SEM Measurements

SEM samples were prepared by dispensing a 15 μl drop of vesicle dispersion on a copper grid and removing the excess solution with filter paper after 30 min. The copper grid was sputtered with a thin layer of gold (JFC-1200 coater, JEOL) before imaging.

### DLS Measurements

Dynamic light scattering (DLS) measurements for individual ABA vesicles were carried out with Brookhaven BI-APD at a 90° angle with 633 nm laser wavelength. DLS measurements for individual PS-PIAT vesicles and multicompartmentalized vesicles were carried out at a 90° angle with 488 nm laser wavelength. All measurements were analyzed using CONTIN analysis.

### Time-Resolved Scanning Confocal Fluorescence Microscopy Imaging

Fluorescence images were obtained using a time-resolved scanning confocal microscope MicroTime 200 (PicoQuant, Berlin). The microscope was equipped with a 100× objective (Plan-Apo, NA=1.4, optimized for 400-850 nm), nanosecond pulsed laser light sources emitting at 470 (LDH-D-C-470, PicoQuant, Berlin) and 640 nm (LDH-D-C-640, PicoQuant, Berlin), suitable optical filters and dichroic mirrors, and avalanche photodiodes as photon detectors.

The samples for microscopy were prepared by adding 500 μl of diluted vesicle (1:100) solution onto a glass cover slip for few seconds to allow the vesicles to adhere onto the surface. Excess solution was removed by a pipette. The concentration of the vesicle solutions were adjusted so that a surface coverage of less than 10% was obtained, resulting in an average vesicle separation above the optical diffraction limit imposed by the microscope imaging system. The excitation power was adjusted depending on the concentration of the chromophores in the vesicles to minimize photobleaching.

### Flow Cytometry Measurements

Polymersomes were analyzed using BD FACSCalibur (without sorter). Calcein was detected using a 530±30 nm bandpass filter. Cy5-IgG was detected using a 650 nm long pass filter. Data was presented as a two dimensional dot plot between calcein and Cy5-IgG using forward- and side-angle scatter (FSC/SSC) gating to exclude larger particles and noise from the system.

### Results and Discussions

To demonstrate the multicompartmentalization and encapsulation concept, two different block copolymers were used, namely ABA and PS-PIAT. ABA polymersomes were obtained by the film rehydration method and PS-PIAT polymersomes by direct dissolution method. For double encapsulation, PS-PIAT polymer in THF solution was added into the aqueous phase containing ABA polymersomes.

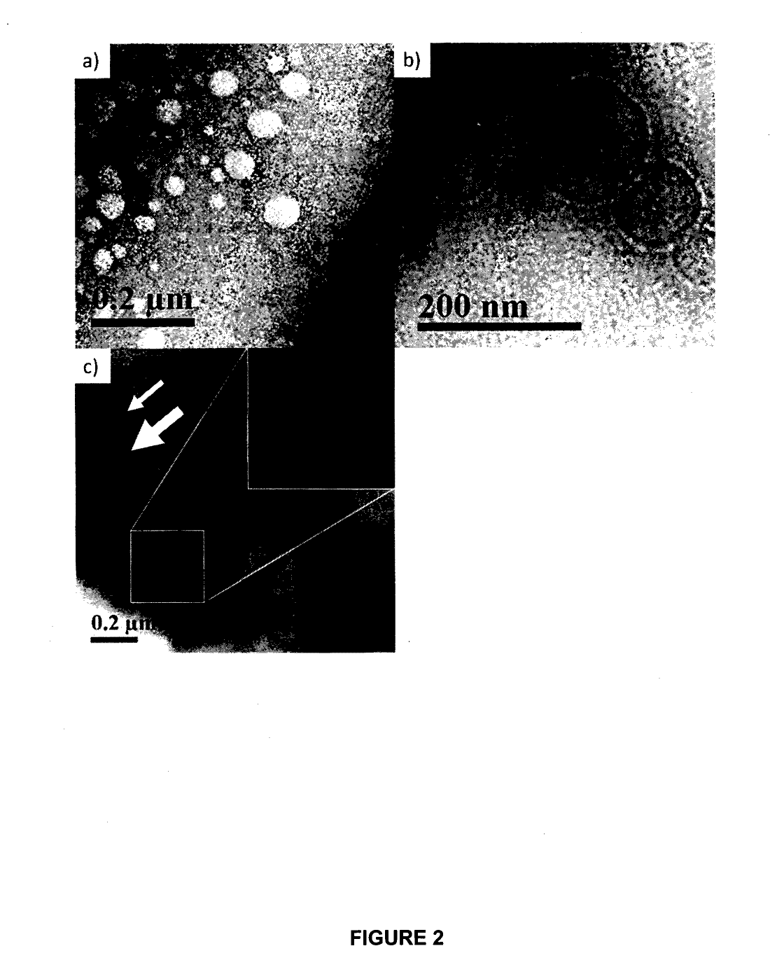

Multicompartmentalized polymersomes as well as single polymersomes formed from the individual copolymers (PS-PIAT and ABA) were studied with transmission electron microscopy (TEM). Images of the individual polymersomes confirm their respective hollow shell structure (FIGS. 2a and 2b). TEM image of the multicompartmentalized polymersomes (FIG. 2c) shows the presence of three populations of vesicles: individual ABA and PS-PIAT polymersomes (FIG. 2c; thin and thick arrows respectively), and polymersomes with a dark core structure. These dark core structures are the ABA polymersomes encapsulated within PS-PIAT polymersomes, i.e., multicompartmentalized polymersomes (FIG. 2c; inset). The TEM images further suggest that each vesicle preparation (ABA, PS-PIAT and multicompartmentalized) had distinctly different sizes; i.e., individual ABA polymersomes had the smallest diameter and multicompartmentalized polymersomes had the largest.

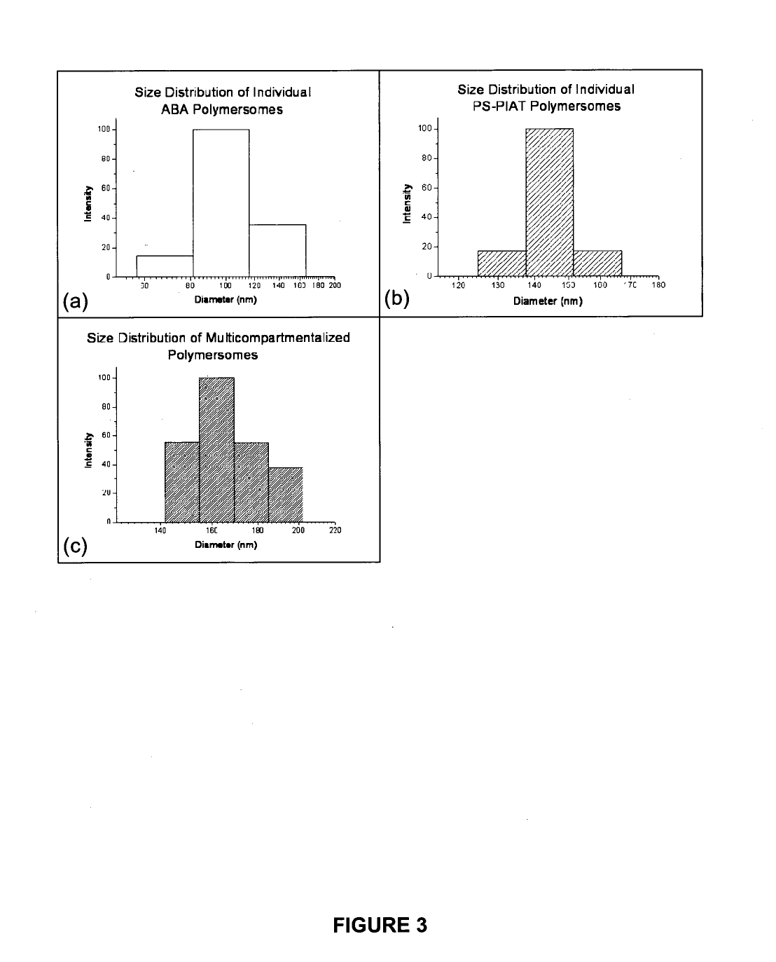

Dynamic light scattering (DLS) measurements were performed to confirm this trend (FIG. 3). The measurements show that individual ABA vesicles averaged about 100 nm in diameter, individual PS-PIAT vesicles about 145 nm and multicompartmentalized vesicles about 160 nm. The self-assembly of PS-PIAT polymersomes have been observed to be affected by particles present in the buffer solution, and that the size of PS-PIAT polymersomes in the multicompartmentalized structure is at least in part affected by the size of the particles in the buffer (in this case the ABA polymersomes).

Different protein species were encapsulated inside the respective vesicles. As control experiments, GFP was encapsulated inside ABA polymersomes and Cy5-IgG was encapsulated inside PS-PIAT polymersomes. In both cases, the non-encapsulated proteins were removed as described above.

For multicompartmentalization or double encapsulation vesicular structure, the PS-PIAT copolymer in THF was added to a solution containing Cy5-IgG and ABA vesicles encapsulated with calcein to demonstrate selective encapsulation. After purification by filtration, the samples were analyzed under SEM and fluorescence microscopy.

The resulting individual and multicompartmentalized polymersomes were characterized by wavelength resolved scanning confocal microscopy. Fluorescence measurements were performed at different excitation wavelengths to selectively excite and detect the emission from GFP (lex=395 nm, lem=509 nm) and Cy5-IgG (lex=649 nm, lem=670 nm).

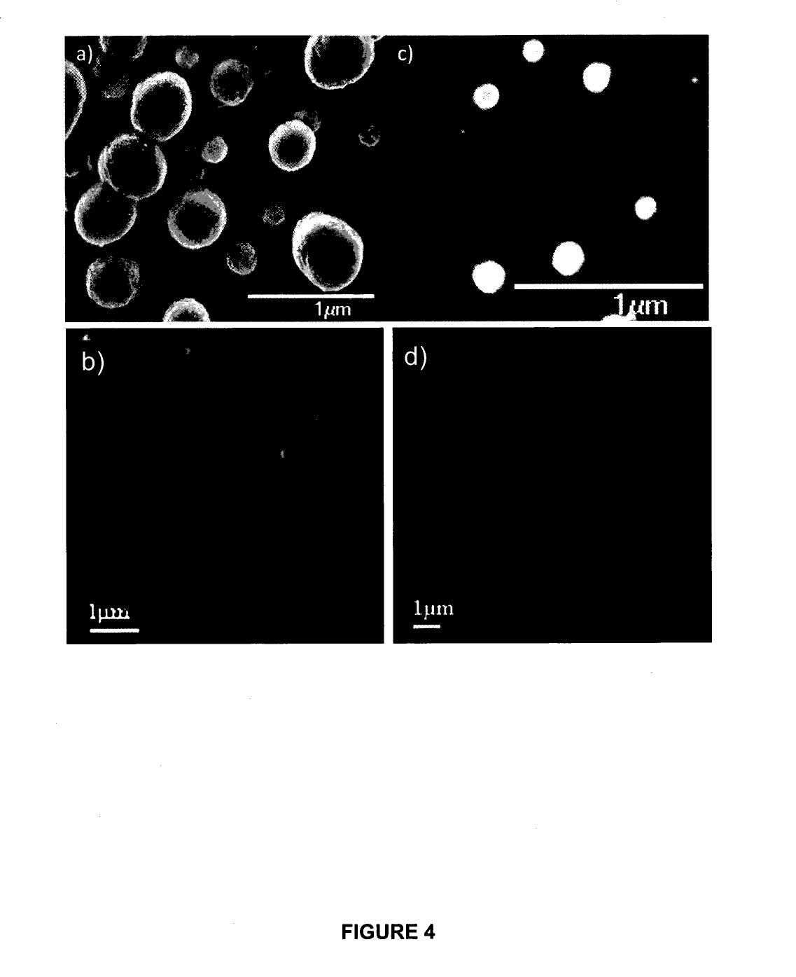

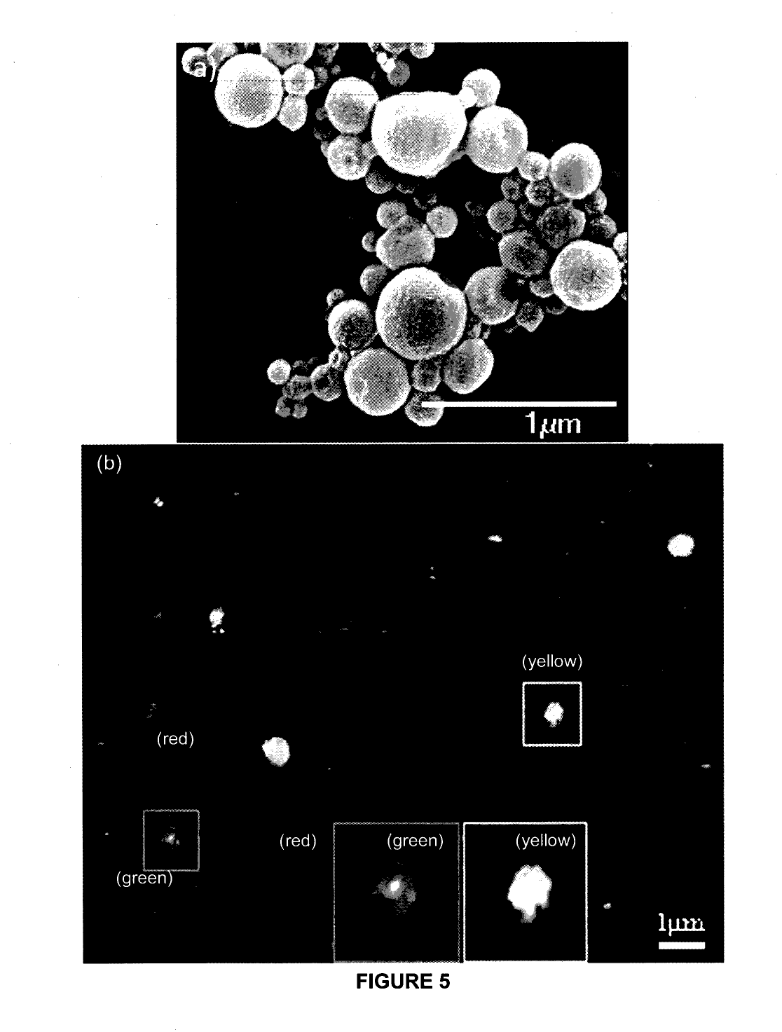

SEM and fluorescence images in FIG. 4 depict the individual control vesicles and their corresponding fluorescence of the encapsulated proteins. SEM and fluorescence images in FIG. 5 depict the multicompartmenalized vesicular structure and its corresponding fluorescence of the selectively encapsulated proteins. Microscopy images show the presence of GFP in individual ABA vesicles (green spots in FIG. 4b) and Cy5-IgG in individual PS-PIAT vesicles (red spots in FIG. 4d).

The surface of the structure of double encapsulated vesicles shown in FIG. 5a is comparable with that of PS-PIAT vesicles (FIG. 4c). This clearly shows that PS-PIAT is the outer vesicle. Furthermore, the fluorescence image in FIG. 5b is obtained by super-imposing two images taken at different excitation wavelengths (for calcein at 470 nm and for Cy5 at 640 nm). Co-localization of the respective emissions of FIG. 4b (green) and 4d (red) (resulting in the yellow spots in FIG. 5b) clearly reveals the presence of the two fluorophores within the same multicompartmentalized polymersomes. The relatively low number of individual PS-PIAT polymersomes may be due to the effect of the particles in solution on the self-assembly of PS-PIAT polymersomes, as mentioned earlier. It clearly demonstrates that double encapsulated structures has been obtained. In both images, well-formed, round and clearly luminescent vesicles are observed.

To rule out the possibility that both GFP and Cy5-IgG were encapsulated in a single compartment (i.e., in the intra-vesicular space within PS-PIAT polymersomes but outside of the ABA polymersomes), GFP was replaced with calcein.

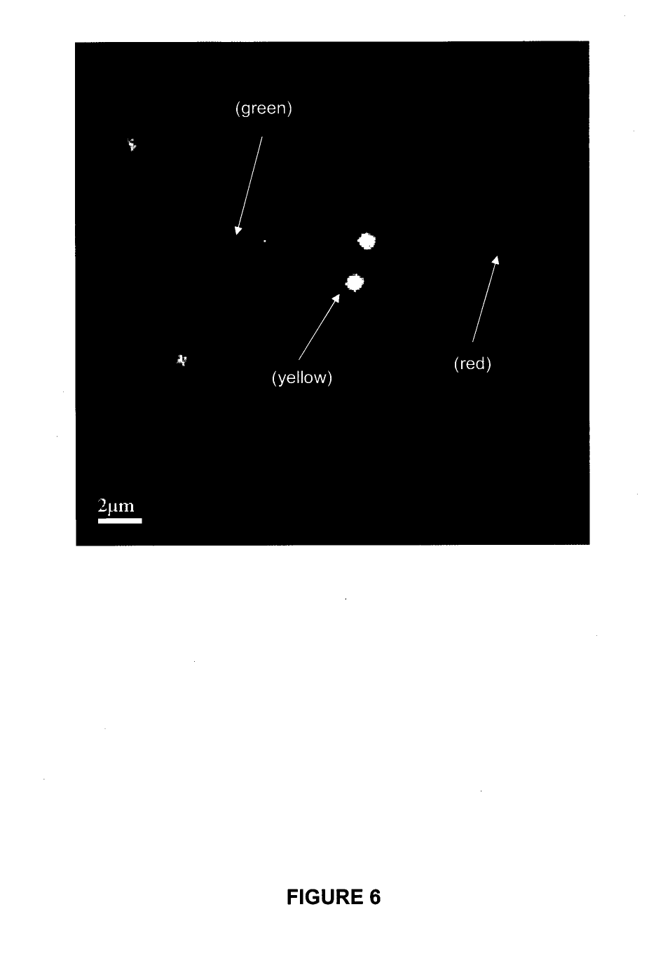

Calcein is a fluorescent dye (lex=495 nm, lem=515 nm) with a molecular weight of 622 g mol⁻¹ that is small enough to be able to diffuse through PS-PIAT membranes but not through ABA membranes, which are tightly packed. If calcein was encapsulated by PS-PIAT polymersomes, it would leak into the buffer solution after an extensive filtration process and no fluorescent signal from calcein would be detected from the polymersomes. Hence, no co-localization of calcein and Cy5-IgG would occur. Conversely, calcein would remain trapped within ABA polymersomes even after filtration, resulting in co-localized signals with Cy5-IgG. Such co-localization of calcein and Cy5-IgG emission did occur in accordance with a multicompartmentalized structure, and it is concluded that the dyes were encapsulated in different separate compartments (FIG. 6).

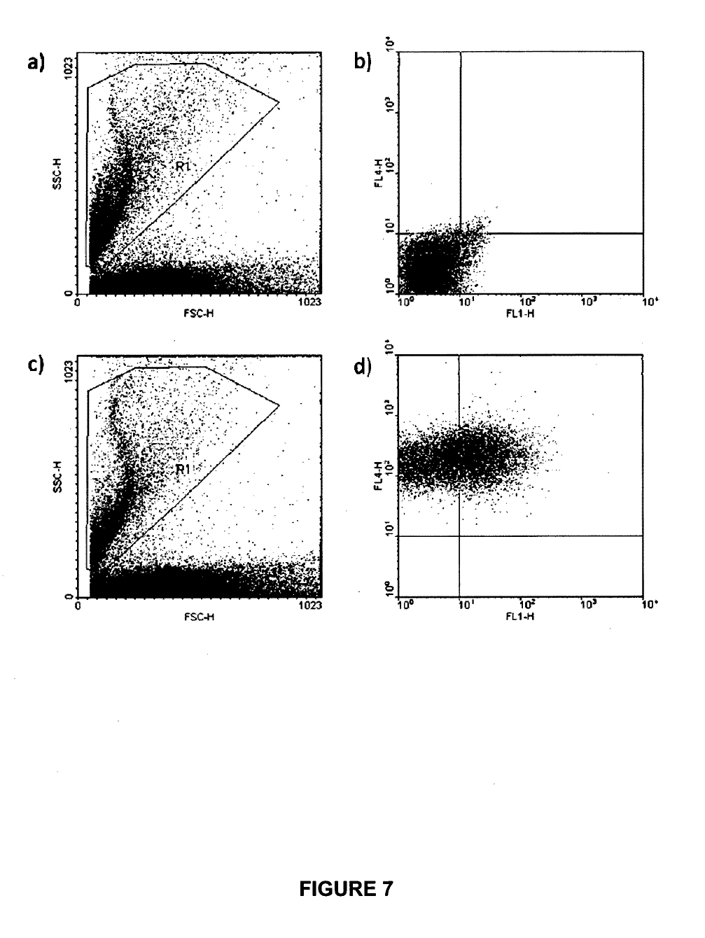

Flow cytometry was used to estimate the efficiency of the double-encapsulation procedure (FIG. 7). The fraction of multicompartmentalized polymersomes with calcein and Cy5-IgG encapsulated into separate compartments was found to be equal to 45% compared to the whole polymersome population. The absence of ABA polymersomes encapsulating calcein, as observed by flow cytometry, is likely to be the result of the centrifugation process, where the size cut-off of the filters used was similar to the diameter of the ABA polymersomes (100 nm).

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

1. A method for forming a multicompartmentalized vesicular structure comprising an outer block copolymer vesicle and at least one inner block copolymer vesicle, wherein the at least one inner block copolymer vesicle is encapsulated inside the outer block copolymer vesicle, the method comprising:
(a) forming the at least one inner block copolymer vesicle by any method; and
(b) forming the outer block copolymer vesicle using a direct dissolution method by adding block copolymers dissolved in a suitable solvent to a dispersion of the at least one inner block copolymer vesicle in an aqueous buffer under conditions that allow the block copolymers to form the outer block copolymer vesicle and encapsulate the at least one inner block copolymer vesicle.

2. The method of claim 1, wherein the block copolymer forming the outer block copolymer vesicle and the block copolymer forming the at least one inner block copolymer vesicle are the same or different.

3. The method of claim 1, wherein the outer block copolymer vesicle, the at least one inner block copolymer vesicle, or both are formed by self-assembly of the block copolymers.

4. The method of claim 1, wherein the at least one inner block copolymer vesicle comprises at least two block copolymer vesicles that are the same or different.

5. The method of claim 1, wherein the outer block copolymer vesicle is a polymersome formed of an amphiphilic diblock, triblock, tetrablock or pentablock copolymer.

6. The method of claim 5, wherein the outer block copolymer vesicle is a polymersome formed of a copolymer selected from the group consisting of poly[styrene-b-poly(L-isocyanoalanine(2-thiophen-3-yl-ethyl)amide)] (PS-PIAT), poly(butadiene)-poly(ethylene oxide) (PBD-PEO), poly(ethylene oxide)-poly(caprolactone) (PEO-PCL), poly(ethyl ethylene)-poly(ethylene oxide) (PEE-PEO), poly(ethylene oxide)-poly(lactic acid) (PEO-PLA), poly(isoprene)-poly(ethylene oxide) (PI-PEO), poly(2-vinylpyridine)-poly(ethylene oxide) (P2VP-PEO), poly(ethylene oxide)-poly(N-isopropylacrylamide) (PEO-PNIPAm), poly(styrene)-poly(acrylic acid) (PS-PAA), poly(ethylene glycol)-poly(propylene sulfide) (PEG-PPS), poly(2-methyloxazoline)-poly(dimethylsiloxane)-poly(2-methyloxazoline) (PMOXA-PDMS-PMOXA), poly(ethylene oxide)-poly(dimethylsiloxane)-poly(2-methyloxazoline) (PEO-PDMS-PMOXA) and poly(methylphenylsilane)-poly(ethylene oxide) (PMPS-PEO-PMPS-PEO-PMPS).

7. The method of claim 1, wherein the at least one inner block copolymer vesicle is a polymersome formed of an amphiphilic diblock, triblock, tetrablock or pentablock copolymer.

8. The method of claim 7, wherein the at least one inner block copolymer vesicle is a polymersome formed of a copolymer selected from the group consisting of poly[styrene-b-poly(L-isocyanoalanine(2-thiophen-3-yl-ethyl)amide)] (PS-PIAT), poly(butadiene)-poly(ethylene oxide) (PBD-PEO), poly(ethylene oxide)-poly(caprolactone) (PEO-PCL), poly(ethyl ethylene)-poly(ethylene oxide) (PEE-PEO), poly(ethylene oxide)-poly(lactic acid) (PEO-PLA), poly(isoprene)-poly(ethylene oxide) (PI-PEO), poly(2-vinylpyridine)-poly(ethylene oxide) (P2VP-PEO), poly(ethylene oxide)-poly(N-isopropylacrylamide) (PEO-PNIPAm), poly(styrene)-poly(acrylic acid) (PS-PAA), poly(ethylene glycol)-poly(propylene sulfide) (PEG-PPS), poly(2-methyloxazoline)-poly(dimethylsiloxane)-poly(2-methyloxazoline) (PMOXA-PDMS-PMOXA), poly(ethylene oxide)-poly(dimethylsiloxane)-poly(2-methyloxazoline) (PEO-PDMS-PMOXA) and poly(methylphenylsilane)-poly(ethylene oxide) (PMPS-PEO-PMPS-PEO-PMPS).

9. The method of claim 1, wherein the at least one inner block copolymer vesicle is formed by:
(a) dissolving block copolymers in a suitable solvent;
(b) drying the solution of (a) to obtain a polymer film; and
(c) rehydrating the polymer film of (b) in an aqueous buffer.

10. The method of claim 1, wherein the outer block copolymer vesicle, the at least one inner block copolymer vesicle, or both include at least one substance encapsulated inside the respective vesicle, wherein the at least one substance is selected from the group consisting of organic molecules, biomolecules and ions or wherein the at least one substance is a marker substance selected from the group consisting of fluorophores, chromophores, radiomarkers, fluorescent proteins, enzymes, and fluorophore-, chromophore- or radio-labelled proteins.

11. The method of claim 10, wherein the at least one substance is selected from the group consisting of organic molecules, biomolecules and ions.

12. The method of claim 11, wherein the biomolecules comprise proteins, lipids, carbohydrates, and nucleic acids.

13. The method of claim 10, wherein the at least one substance is a marker substance selected from the group consisting of fluorophores, chromophores, radiomarkers, fluorescent proteins, enzymes, and fluorophore-, chromophore- or radio-labelled proteins.

14. The method of claim 10, wherein the at least one inner block copolymer vesicle encapsulating the at least one substance is obtained by:
(a) dissolving block copolymers in a suitable solvent;
(b) drying the solution of (a) to obtain a polymer film;
(c) rehydrating the polymer film of (b) in an aqueous buffer containing the at least one substance.

15. The method of claim 10, wherein the outer block copolymer vesicle encapsulating the at least one substance is obtained by adding block copolymers dissolved in a suitable solvent to a dispersion of the at least one inner block copolymer vesicle in an aqueous buffer under conditions that allow the block copolymers to form the outer block copolymer vesicle and encapsulate the at least one inner block copolymer vesicle, wherein the at least one substance is added (i) to the solution of the block copolymers or (ii) the dispersion of the at least one inner block copolymer vesicle before adding the solution of the block copolymers.

16. The method of claim 1, wherein the solvent for dissolving the block copolymer of the outer vesicle is selected from the group consisting of tetrahydrofuran (THF), ethanol, dimethylsulfoxide, dimethylformamide, dioxane, and water.

17. (canceled)

18. The method of claim 1, wherein the aqueous buffer is a buffered solution or buffers with detergents.

19. The method of claim 18, wherein the buffered solution is selected from the group consisting of phosphate buffered saline (PBS), Tris buffered saline (TBS), Hank's balanced salt solution (HBSS), Earle's balanced salt solution (EBSS), Standard saline citrate (SSC), HEPES-buffered saline (HBS), and Grey's balanced salt solution (GBSS).

20. The method of claim 18, wherein the buffers are formed of sodium carbonate, N-(2-Hydroxyethyl)-piperazine-N′-ethanesulfonic acid (HEPES), 3-(N-Morpholino)-propanesulfonic acid, Tris(hydroxymethyl)-aminomethane (Tris), phosphates.

21. The method of claim 1, wherein allowing the block copolymers to form the outer block copolymer vesicle comprises incubating the reaction mixture at least 10 min.

22. The method of claim 1, wherein the outer block copolymer vesicle, the at least one inner block copolymer vesicle, or both comprise at least one molecule that modulates vesicular membrane permeability.

23. The method of claim 22, wherein the molecule is present in the vesicular membrane.

24. The method of claim 23, wherein the molecule is selected from the group of transmembrane proteins, membrane associated proteins, and lipids.

25. The method of claim 24, wherein the molecule is a transporter molecule or ion channel.

26. A multicompartmentalized vesicular structure comprising an outer block copolymer vesicle and at least one inner block copolymer vesicle, wherein the at least one inner block copolymer vesicle is encapsulated inside the outer block copolymer vesicle, and wherein the block copolymer forming the outer block copolymer vesicle and the block copolymer forming the at least one inner block copolymer vesicle are different.

27. A multicompartmentalized vesicular structure comprising an outer block copolymer vesicle and at least one inner block copolymer vesicle, wherein the at least one inner block copolymer vesicle is encapsulated inside the outer block copolymer vesicle, and wherein both the outer block copolymer vesicle and the at least one inner block copolymer vesicle include at least one substance encapsulated inside the respective vesicle.

28. The multicompartmentalized vesicular structure of claim 27, wherein the block copolymer forming the outer block copolymer vesicle and the block copolymer forming the at least one inner block copolymer vesicle are the same or different.

29. The multicompartmentalized vesicular structure of claim 27, wherein the at least one inner block copolymer vesicle comprises at least two block copolymer vesicles that are the same or different.

30. The multicompartmentalized vesicular structure of claim 27, wherein the outer block copolymer vesicle is a polymersome formed of an amphiphilic diblock, triblock, tetrablock or pentablock copolymer.

31. The multicompartmentalized vesicular structure of claim 30, wherein the outer block copolymer vesicle is a polymersome formed of a copolymer selected from the group consisting of poly[styrene-b-poly(L-isocyanoalanine(2-thiophen-3-yl-ethyl)amide)] (PS-PIAT), poly(butadiene)-poly(ethylene oxide) (PBD-PEO), poly(ethylene oxide)-poly(caprolactone) (PEO-PCL), poly(ethyl ethylene)-poly(ethylene oxide) (PEE-PEO), poly(ethylene oxide)-poly(lactic acid) (PEO-PLA), poly(isoprene)-poly(ethylene oxide) (PI-PEO), poly(2-vinylpyridine)-poly(ethylene oxide) (P2VP-PEO), poly(ethylene oxide)-poly(N-isopropylacrylamide) (PEO-PNIPAm), poly(styrene)-poly(acrylic acid) (PS-PAA), poly(ethylene glycol)-poly(propylene sulfide) (PEG-PPS), poly(2-methyloxazoline)-poly(dimethylsiloxane)-poly(2-methyloxazoline) (PMOXA-PDMS-PMOXA), poly(ethylene oxide)-poly(dimethylsiloxane)-poly(2-methyloxazoline) (PEO-PDMS-PMOXA) and poly(methylphenylsilane)-poly(ethylene oxide) (PMPS-PEO-PMPS-PEO-PMPS).

32. The multicompartmentalized vesicular structure of claim 27, wherein the at least one inner block copolymer vesicle is a polymersome formed of an amphiphilic diblock, triblock, tetrablock or pentablock copolymer.

33. The multicompartmentalized vesicular structure of claim 32, wherein the at least one inner block copolymer vesicle is a polymersome formed of acopolymer selected from the group consisting of poly[styrene-b-poly(L-isocyanoalanine(2-thiophen-3-yl-ethyl)amide)] (PS-PIAT), poly(butadiene)-poly(ethylene oxide) (PBD-PEO), poly(ethylene oxide)-poly(caprolactone) (PEO-PCL), poly(ethyl ethylene)-poly(ethylene oxide) (PEE-PEO), poly(ethylene oxide)-poly(lactic acid) (PEO-PLA), poly(isoprene)-poly(ethylene oxide) (PI-PEO), poly(2-vinylpyridine)-poly(ethylene oxide) (P2VP-PEO), poly(ethylene oxide)-poly(N-isopropylacrylamide) (PEO-PNIPAm), poly(styrene)-poly(acrylic acid) (PS-PAA), poly(ethylene glycol)-poly(propylene sulfide) (PEG-PPS), poly(2-methyloxazoline)-poly(dimethylsiloxane)-poly(2-methyloxazoline) (PMOXA-PDMS-PMOXA), poly(ethylene oxide)-poly(dimethylsiloxane)-poly(2-methyloxazoline) (PEO-PDMS-PMOXA) and poly(methylphenylsilane)-poly(ethylene oxide) (PMPS-PEO-PMPS-PEO-PMPS).

34. The multicompartmentalized vesicular structure of claim 27, wherein the at least one substance is selected from the group consisting of organic molecules, biomolecules and ions.

35. The multicompartmentalized vesicular structure of claim 34, wherein the biomolecules comprise proteins, lipids, carbohydrates, and nucleic acids.

36. The multicompartmentalized vesicular structure of claim 27, wherein the at least one substance is a marker substance selected from the group consisting of fluorophores, chromophores, radiomarkers, fluorescent proteins, enzymes, and fluorophore-, chromophore- or radio-labelled proteins.

37. The multicompartmentalized vesicular structure of claim 27, wherein the outer block copolymer vesicle, the at least one inner block copolymer vesicle, or both comprise at least one molecule that modulates vesicular membrane permeability.

38. The multicompartmentalized vesicular structure of claim 37, wherein the molecule is present in the vesicular membrane.

39. The multicompartmentalized vesicular structure of claim 38, wherein the molecule is selected from the group of transmembrane proteins, membrane associated proteins, and lipids.

40. The multicompartmentalized vesicular structure of claim 39, wherein the molecule is a transporter molecule or ion channel.

41. Use of the multicompartmentalized vesicular structure of claim 27 for selective encapsulation of different substances, wherein the inner block copolymer vesicle encapsulates a first substance and the outer block copolymer vesicle encapsulates a second substance and the inner block copolymer vesicle.

42. The use of claim 41 for controlled release or delivery of the different substances encapsulated in the respective inner block copolymer vesicle and outer block copolymer vesicle.

43. The use of claim 42, wherein the controlled release or delivery is triggered by a stimulant.

44. The use of claim 43, wherein the stimulant is light, temperature, pH, electric field, magnetic field, bacteria, viruses, pathogens, or chemical compounds.

45. A method of releasing or delivering substances encapsulated in the respective inner block copolymer vesicle and outer block copolymer vesicle of the multicompartmentalized vesicular structure of claim 27, wherein the method comprises contacting the multicompartmentalized vesicular structure with a cell so that the multicompartmentalized vesicular structure is taken up into the cell.

46. The method of claim 45, wherein the cell is in vitro.

47. The method of claim 45, wherein the cell is in vivo and the method further comprises administering the multicompartmentalized vesicular structure to a subject.

48. The method of claim 47, wherein the subject is a human.

49. A pharmaceutical composition comprising multicompartmentalized vesicular structure of claim 27 and a pharmaceutically acceptable carrier.

50. An in vitro method of identifying a compound capable of forming a complex with a membrane receptor protein, the method comprising:
(a) providing a multicompartmentalized vesicular structure of claim 39, wherein the membrane associated protein is the membrane receptor protein;
(b) contacting the multicompartmentalized vesicular structure with a candidate compound suspected to be capable of forming a complex with the membrane receptor protein; and
(c) detecting the said complex formation.

51. An in vitro method of identifying a compound capable of modulating the function of an ion channel protein, wherein the ion channel protein is capable of allowing a known ion to pass, the method comprising:
(a) providing a multicompartmentalized vesicular structure of claim 40, wherein the membrane protein is the ion channel protein;
(b) contacting the multicompartmentalized vesicular structure with a candidate compound suspected to modulate the function of the ion channel protein; and
(c) detecting the passage of ions into or out of the multicompartmentalized vesicular structure.

52. The method of claim 50 or 51, wherein the multicompartmentalized vesicular structure is immobilized on a surface.
